(19) United States
(12) Patent Application Publication
(10) Pub. No.: US 2024/0279212 A1
(43) Pub. Date: Aug. 22, 2024

(54) NOVEL INTERMEDIATE FOR PREPARATION OF PYROXASULFONE

(71) Applicant: UPL LIMITED, Mumbai (IN)

(72) Inventors: Prashant Vasant KINI, Mumbai (IN); Sopan Nagnath GANDHALE, Mumbai (IN); Debasish SENGUPTA, Mumbai (IN); Sandip Sahebrao GULVE, Mumbai (IN); Vijay MAHAJAN, Mumbai (IN)

(21) Appl. No.: 18/562,483

(22) PCT Filed: May 27, 2022
(86) PCT No.: PCT/IN2022/050495

§ 371 Date: Nov. 20, 2023

(30) Foreign Application Priority Data
May 27, 2021 (IN) 202121023677

(51) Int. Cl. C07D 413/12; A01N 25/14; A01N 43/80; A01P 13/00; C07D 231/16

(52) U.S. Cl. CPC C07D 413/12; A01N 25/14; A01N 43/80; A01P 13/00; C07D 231/16

(57) ABSTRACT

The present invention relates to a novel a compound of formula (I) or its salt.

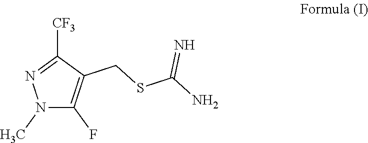 [SMILES: Cn1nc(C(F)(F)F)c(CSC(=N)N)c1F]

The present invention further relates to process for preparation of Pyroxasulfone using said compound of formula (I) or its salt.

### FIELD OF THE INVENTION

The present invention relates to a novel intermediate of the formula (I) or its salt, which is a valuable intermediate in synthesis of Pyroxasulfone and process for preparation of the same. The present invention further relates to a process for preparation of Pyroxasulfone using this novel intermediate of formula (I) or its salt.

### BACKGROUND OF THE INVENTION

Pyroxasulfone is a herbicide belonging to the group of pyrazolium. Pyroxasulfone is chemically known as 3-[5-(difluoromethoxy)-1-methyl-3-(trifluoromethyl)pyrazol-4-ylmethylsulfonyl]-4,5-dihydro-5,5-dimethyl-1,2-oxazole and represented by compound of formula (VII).

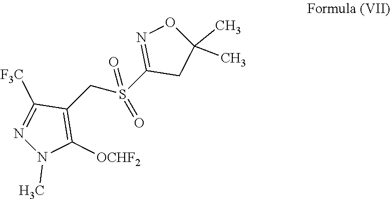 [SMILES: Cn1nc(C(F)(F)F)c(CS(=O)(=O)C2=NOC(C)(C)C2)c1OC(F)F]

It is a pre-emergence herbicide that inhibits the biosynthesis of very long chain fatty acids. It can be used to effectively control grass and broad-leaved weeds in corn, soybean, and wheat fields.

Pyroxasulfone was first disclosed in U.S. Pat. No. 7,238,689. Currently, few processes for preparation of Pyroxasulfone are known.

The method for preparation of Pyroxasulfone known in the prior art are lengthy, tedious and possess problems in the scale-up to commercial production. Another issue involved is availability of the starting material used. These problems have necessitated further research in an attempt to discover a shorter route which involves use of easily available materials for preparation of Pyroxasulfone.

The inventors of present invention have now evolved a synthetic route for the preparation of Pyroxasulfone which starts from easily available materials and employs mild reaction conditions and has simpler after-treatment procedures, thus making it suitable for large-scale production. Certain alternatives are available in the early stages of this route, which is indeed advantageous since it opens the way to the use of different reaction strategies. All these alternative routes however pass through the same novel pyrazole intermediate of formula (Ia).

### OBJECT OF THE INVENTION

It is an object of the present invention to provide a compound of formula (Ia) or its salt.

It is an object of the present invention to provide a compound of formula (I) or its salt.

Another object of the present invention is to provide a process for preparation of a compound of formula (I) or its salt.

Yet another object of the present invention is to provide an alternative synthesis route for preparation of Pyroxasulfone using compound of formula (I) or its salt.

Yet another object of the present invention is to provide an alternative synthesis route for preparation of compound of formula (IV), an advance intermediate formed in Pyroxasulfone preparation.

### SUMMARY OF THE INVENTION

According to an aspect of the present invention, there is provided a compound of formula (Ia) or its salt.

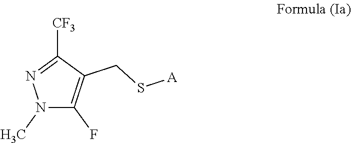

wherein A is hydrogen, —C(NH)(NH₂) or alkali metal.

According to another aspect of the present invention, there is provided a compound of formula (I) or its salt.

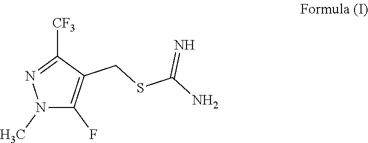 [SMILES: Cn1nc(C(F)(F)F)c(CSC(=N)N)c1F]

According to another aspect of present invention, there is provided a process for the preparation of the compound of formula (I) or its salt, comprising condensing compound of formula (II) with thiourea to obtain the compound of formula (I).

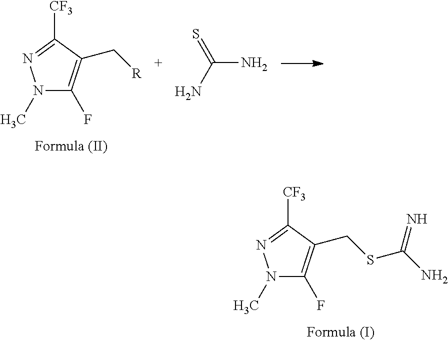

wherein R is selected from halogen or hydroxy group.

According to yet another aspect of the present invention, there is provided a process for preparation of Pyroxasulfone of formula (VII) comprising
a) condensing compound of formula (II)

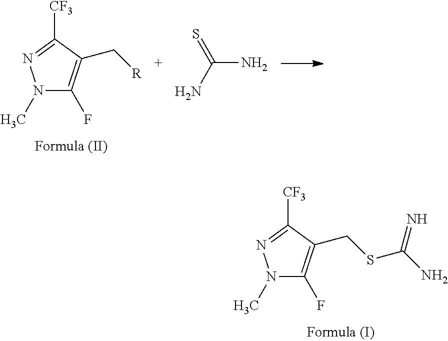

wherein R is selected from halogen or hydroxy group, with thiourea to obtain compound of formula (I) or its salt;
b) condensing compound of formula (I) or its salt with isooxazoline compound of formula (II) to get compound of formula (IV),

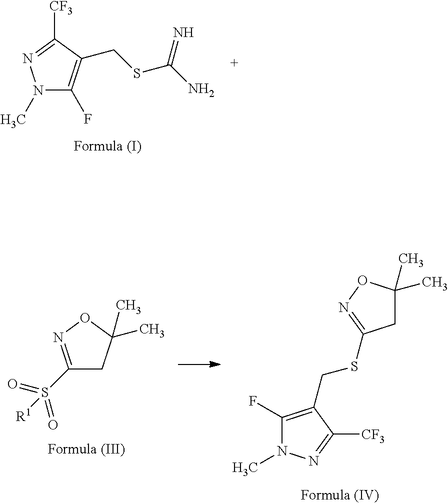

wherein R¹ is lower alkyl group
c) converting compound of formula (IV) to compound of formula (V)

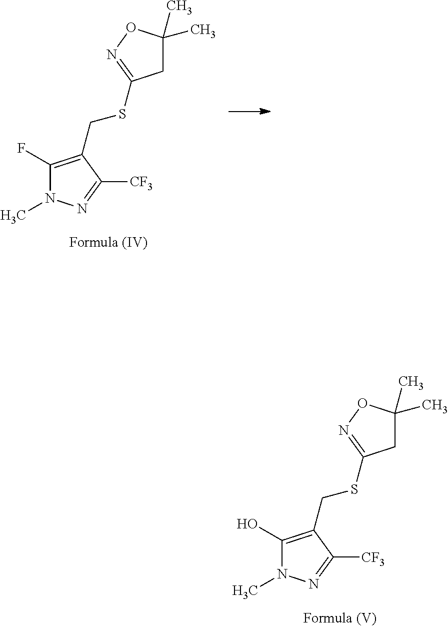 [SMILES: Cn1nc(C(F)(F)F)c(CSC2=NOC(C)(C)C2)c1F ; Cn1nc(C(F)(F)F)c(CSC2=NOC(C)(C)C2)c1O]

d) fluoromethylating compound of formula (V) to get compound of formula (VI)

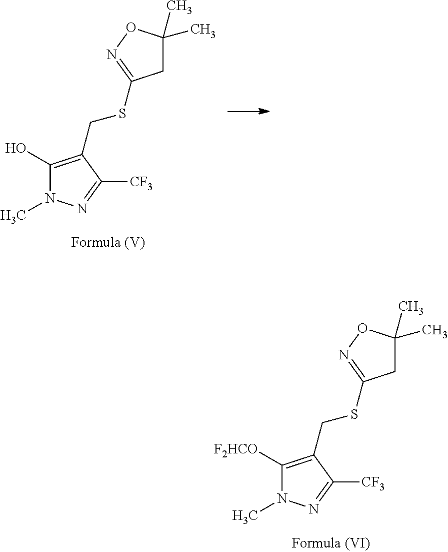 [SMILES: Cn1nc(C(F)(F)F)c(CSC2=NOC(C)(C)C2)c1O ; Cn1nc(C(F)(F)F)c(CSC2=NOC(C)(C)C2)c1OC(F)F]

e) oxidising compound of formula (VI) to get Pyroxasulfone of formula (VII)

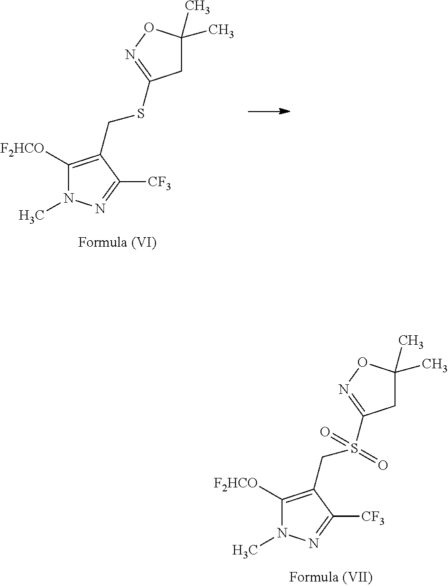 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to yet another aspect of the present invention, there is provided a process for purification of Pyroxasulfone comprising treating the crude Pyroxasulfone with mixture of alcohol and water at temperature ranging from 50 to 110° C.

According to yet another aspect of present invention, there is provided a process for preparation of compound of formula (IV) comprising:
condensing novel compound of formula (Ia) or its salt with isooxazoline compound of formula (IIIa) to get compound of formula (IV);

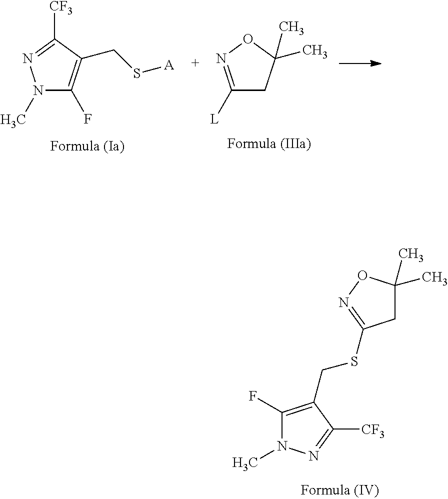

wherein A is hydrogen, —C(NH)(NH₂) or alkali metal, and L is a leaving group.

According to yet another aspect of the present invention, there is provided Pyroxasulfone, is characterized by having D₅₀ particle size value of less than about 200 μm, preferably less than about 150 μm.

According to yet another aspect of the present invention, there is provided Pyroxasulfone, having bulk density of about 0.40 g/cc to 0.90 g/cc.

In another aspect the present invention provides an agrochemical composition comprising Pyroxasulfone prepared according to the present process.

In another aspect the present invention provides an agrochemical composition comprising Pyroxasulfone prepared using the compound of formula (I).

According to yet another aspect of present invention, there is provided a composition comprising Pyroxasulfone characterized by an X-ray powder diffraction pattern exhibiting at least three peaks selected from 9.90°, 17.72°, 17.94°, 19.91°, 20.36°, 20.60°, 21.76°, 22.09°, 22.31°, 22.70°, 25.10°, 25.41°, 26.57°, 27.01°, 28.40°, and 30.18°+0.2° 2θ.

### BRIEF DESCRIPTION OF DRAWING

FIG. 1 illustrates powder X-ray diffraction (PXRD) pattern of Pyroxasulfone prepared according to example 10.

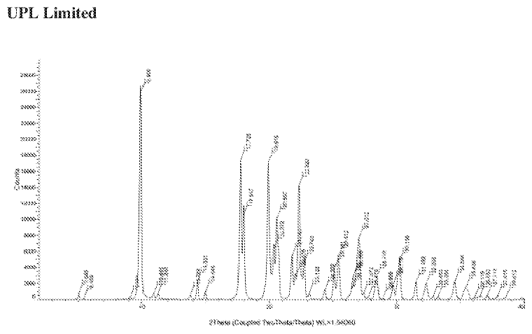

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions, and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or ±5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The term “alkyl” as used herein refers to a straight or branched chain saturated aliphatic hydrocarbon having the specified number of carbon atoms, specifically 1 to 12 carbon atoms, more specifically 1 to 8 carbon atoms.

As used herein, the term “halogen” or “halo” refers to a fluorine, chlorine, bromine, or iodine atom.

The term “room temperature” unless stated otherwise, essentially means temperature in range of 20-45° C.

The term “purity” means purity as determined by HPLC (“High Pressure Liquid Chromatography”).

The term “Pyroxasulfone” or “compound of formula (VII)” as used herein, includes Pyroxasulfone free base or its salts or its crystalline forms and polymorphs and is used interchangeably throughout the disclosure.

The term “its salt” or “salts thereof” are used interchangeably throughout the disclosure.

The present disclosure is not to be limited in scope by the specific embodiments described herein, which are intended for the purposes of exemplification only.

According to an aspect of the present invention, there is provided a compound of formula (Ia) or its salt.

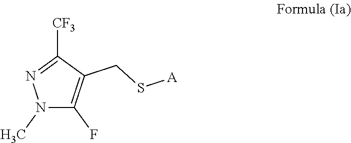

wherein A is hydrogen, —C(NH)(NH₂) or alkali metal.

According to an aspect of the present invention, there is provided a compound of formula (I) or its salt.

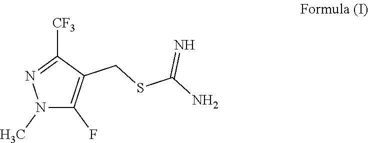 [SMILES: Cn1nc(C(F)(F)F)c(CSC(=N)N)c1F]

According to an embodiment, the salt of compound of formula (I) can be its hydrochloride salt or hydrobromide salt.

According to an embodiment, the salt of compound of formula (I) is its hydrochloride salt.

According to another aspect of present invention, there is provided a process for the preparation of the compound of formula (I) or its salt, comprising condensing compound of formula (II) with thiourea

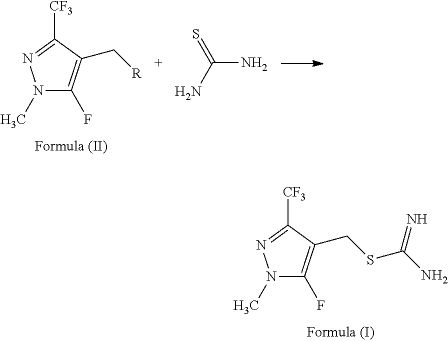

wherein R is selected from halogen or hydroxy group.

According to an embodiment, R is chlorine.

According to an embodiment, R is a hydroxy group.

According to an embodiment, the amount of thiourea used is in the range of 1 to 1.5 moles with respect to the compound of formula (II).

According to an embodiment, the compound of formula (II) is condensed with thiourea in presence of an organic solvent at temperature ranging from 0° C. to 150° C.

The organic solvent used is selected from lower alcohol such as methanol, ethanol, isopropanol, n-propanol, butanol, tert-butanol and the like, hydrocarbons such as toluene, xylene, benzene and like, halogenated hydrocarbons such as dichloromethane, dichloroethane, chloroform and the like, ethers such as methyl tert-butyl ether, tetrahydrofuran, dioxane and the like.

The amount of organic solvent used is in the range of 1 to 60 moles with respect to compound of formula (II).

According to an embodiment, the compound of formula (II) is condensed with thiourea in presence of an organic or inorganic acid.

The organic acid used is selected from acetic acid, formic acid, oxalic acid, and the likes. The inorganic acid used is selected from hydrochloric acid, sulfuric acid, nitric acid, phosphoric acid, boric acid, hydrofluoric acid, hydrobromic acid, perchloric acid, hydroiodic acid.

The amount of acid used may vary from catalytic amount to 6 equivalents with respect to compound of formula (II).

According to an embodiment, the compound of formula (II) is condensed with thiourea at temperature ranging from 0° C. to 150° C. for 0.5 to 20 hours.

According to an embodiment, the compound of formula (I) is isolated by the methods known in prior art for example by filtration, crystallisation, distillation, extraction and the likes.

According to an embodiment, the compound of formula (I) obtained is filtered and washed with a non-polar solvent such as hexane, heptane, petroleum ether or mixture thereof.

According to an embodiment, the compound of formula (I) is in salt form, preferably it is hydrochloride salt.

According to an embodiment, there is provided a process for the preparation of the compound of formula (I) or its salt, comprising condensing compound of formula (IIa) with thiourea.

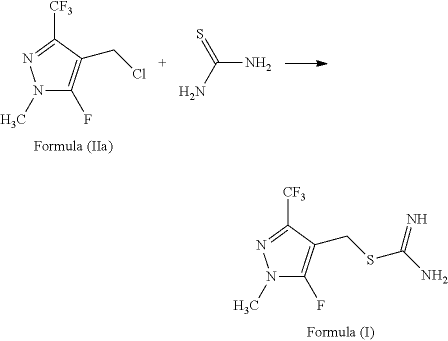

According to another embodiment, there is provided a process for the preparation of the compound of formula (I) or its salt, comprising condensing compound of formula (IIb) with thiourea.

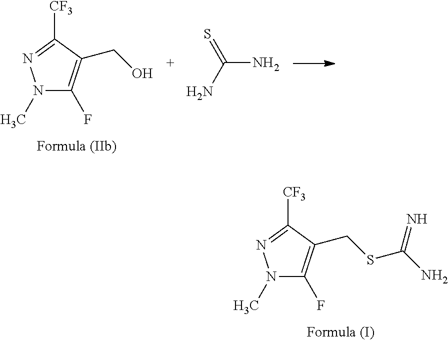

In embodiment, there is provided a process for preparation of Pyroxasulfone of formula (VII) comprising the steps of:
a) condensing compound of formula (II) with thiourea to obtain compound of formula (I) or its salt;

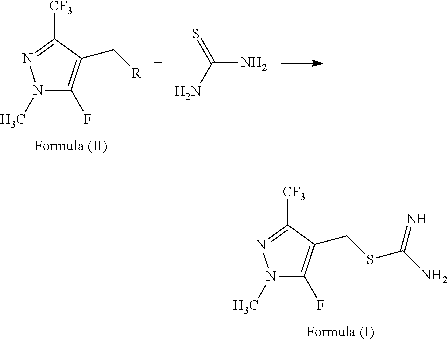

wherein R is selected from halogen or hydroxy group and
b) converting compound of formula (I) or its salt to Pyroxasulfone of formula (VII).

According to an aspect of present invention, there is provided a method of using compound of formula (I) or its salt, in the process for preparation of Pyroxasulfone of formula (VII).

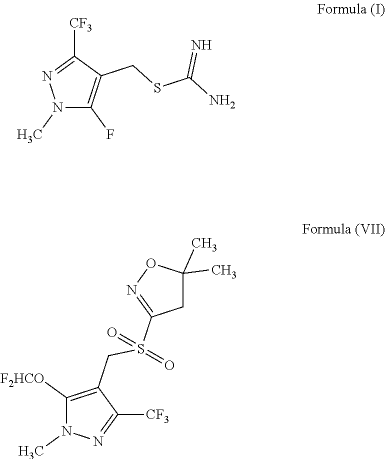 [SMILES: Cn1nc(C(F)(F)F)c(CSC(=N)N)c1F ; Cn1nc(C(F)(F)F)c(CS(=O)(=O)C2=NOC(C)(C)C2)c1OC(F)F]

In an embodiment, the process of converting compound of formula (I) or its salt to Pyroxasulfone of formula (VII) comprising the steps of;
a) condensing compound of formula (I) or its salt with isooxazoline compound of formula (III) to get compound of formula (IV),

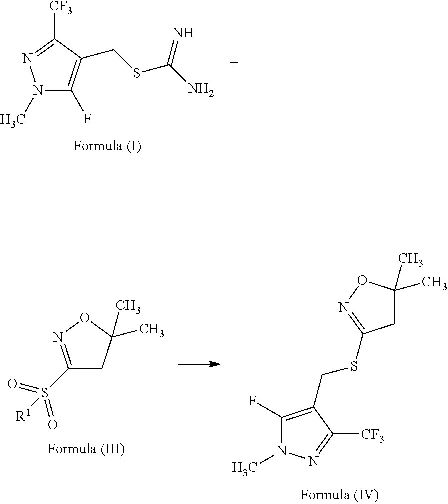

wherein R¹ is lower alkyl group;
b) converting compound of formula (IV) to compound of formula (V);

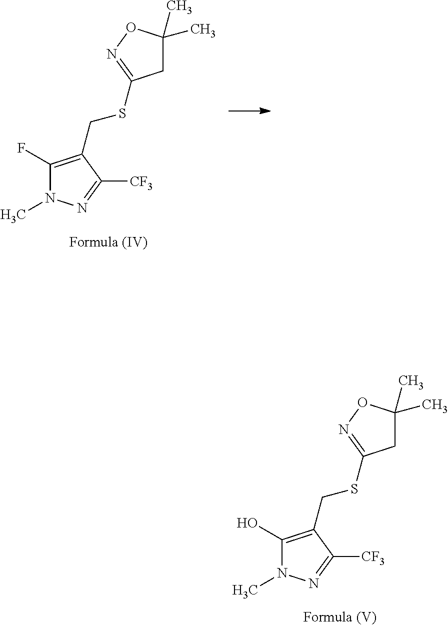 [SMILES: Cn1nc(C(F)(F)F)c(CSC2=NOC(C)(C)C2)c1F ; Cn1nc(C(F)(F)F)c(CSC2=NOC(C)(C)C2)c1O]

c) fluoromethylating compound of formula (V) to get compound of formula (VI) and

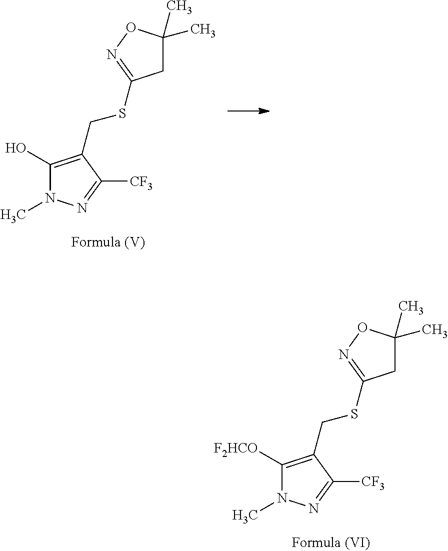 [SMILES: Cn1nc(C(F)(F)F)c(CSC2=NOC(C)(C)C2)c1O ; Cn1nc(C(F)(F)F)c(CSC2=NOC(C)(C)C2)c1OC(F)F]

d) oxidising compound of formula (VI) to get Pyroxasulfone of formula (VII).

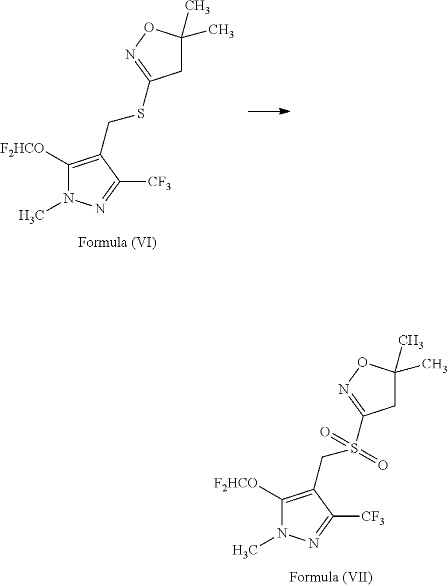 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to an embodiment, there is provided a process for preparation of Pyroxasulfone, wherein the process proceeds via the intermediate of formula (I).

According to an embodiment, there is provided a process for preparation of Pyroxasulfone, wherein the process proceeds via the intermediate of formula (Ia). According to an embodiment, there is provided a process for preparation of Pyroxasulfone, wherein the process proceeds via the intermediate of formula (IV).

According to an embodiment, there is provided a process for preparation of Pyroxasulfone, wherein the process proceeds via [5-Fluoro-1-methyl-3-(trifluoromethyl)-1H-pyrazol-4-yl]methyl carbamimidothioate hydrochloride.

According to yet another aspect of present invention, there is provided a process for preparation of Pyroxasulfone of formula (VII) comprising
a) condensing compound of formula (II) with thiourea to obtain compound of formula (I) or its salt;

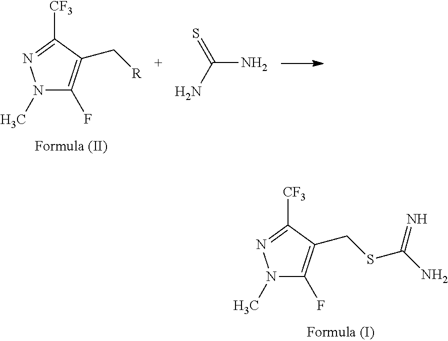

wherein R is selected from halogen or hydroxy group,
b) condensing compound of formula (I) or its salt with isooxazoline compound of formula (III) to get compound of formula (IV),

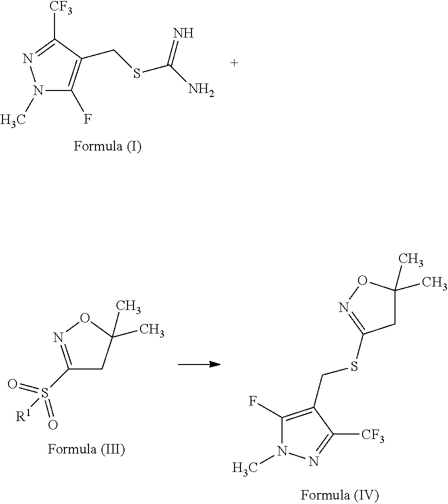

wherein R¹ is lower alkyl group;
c) converting compound of formula (IV) to compound of formula (V);

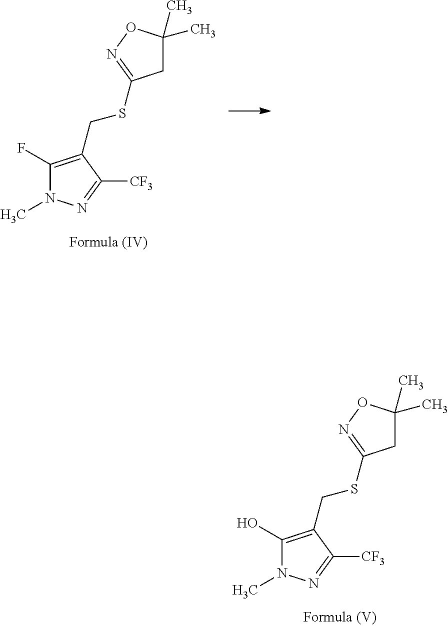 [SMILES: Cn1nc(C(F)(F)F)c(CSC2=NOC(C)(C)C2)c1F ; Cn1nc(C(F)(F)F)c(CSC2=NOC(C)(C)C2)c1O]

d) fluoromethylating compound of formula (V) to get compound of formula (VI) and

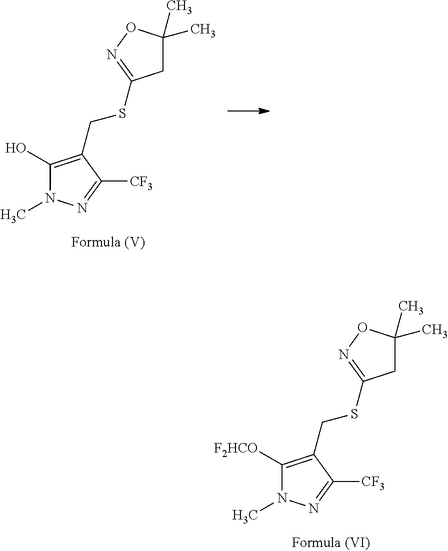 [SMILES: Cn1nc(C(F)(F)F)c(CSC2=NOC(C)(C)C2)c1O ; Cn1nc(C(F)(F)F)c(CSC2=NOC(C)(C)C2)c1OC(F)F]

e) oxidising compound of formula (VI) to get Pyroxasulfone of formula (VII).

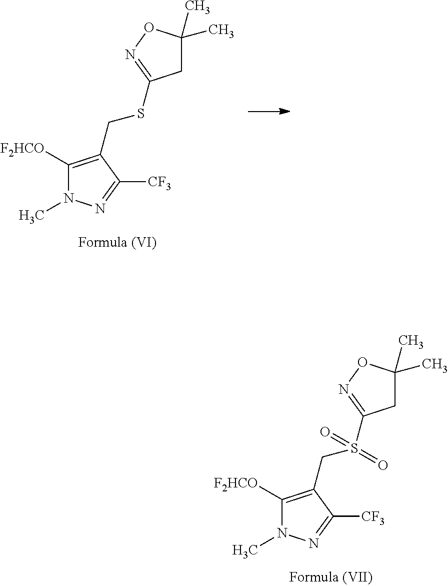 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to an embodiment, the step a) of the process is carried out in presence of an organic solvent at temperature ranging from 0° C. to 150° C.

The organic solvent used is selected from lower alcohol such as methanol, ethanol, isopropanol, n-propanol, butanol, tert-butanol and the like, hydrocarbons such as toluene, xylene, benzene and like, halogenated hydrocarbons such as dichloromethane, dichloroethane, chloroform and the like, ketone solvents such as methyl ethyl ketone, methyl isobutyl ketone and the likes, ethers such as methyl tert-butyl ether, tetrahydrofuran, dioxane and the like.

According to an embodiment, optionally the step a) is carried out in presence of an organic or inorganic acid.

According to another embodiment, the step b) of the process comprises condensing compound of formula (I) or its salt with isooxazoline compound of formula (III) and said step is carried out in presence of a base like carbonates selected from potassium carbonate, potassium bicarbonate, sodium carbonate, sodium bicarbonate and the like, hydroxides selected from potassium hydroxide, sodium hydroxide, ammonium hydroxide and the like, or alkoxides like sodium alkoxide or potassium alkoxide.

The amount of base used is in the range of 0.5 to 3 moles with respect to compound of formula (I).

According to an embodiment, the step b) is carried out in presence of a polar solvent such as alcohols like methanol, ethanol, isopropanol, n-propanol, tert-butanol and the like, ethers such as tetrahydrofuran, 1,6-dioxane and the like, water, dimethylformamide or mixture thereof.

According to an embodiment, an alcohol, water, or a mixture thereof is used as solvent.

In an embodiment, the step b) is carried out at temperature ranging from 0° C. to 150° C.

According to another embodiment, in the step c) of the process comprises converting compound of formula (IV) to compound of formula (V) wherein the compound of formula (IV) is first converted to compound of formula (Va) followed by converting compound of formula (Va) to compound of formula (V).

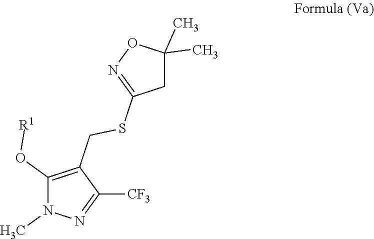

wherein R¹ is lower alkyl group.

According to another embodiment, in the step c) the compound of formula (IV) is alkoxylated using an alkoxylating agent in presence of an alcohol to obtain compound of formula (Va); followed by treatment with an acid to obtain compound of formula (V).

The reaction can be represented as in Scheme (I)

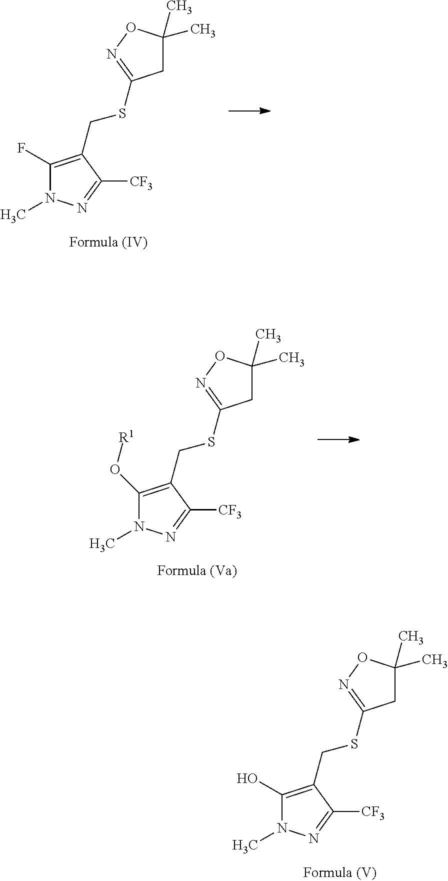

wherein R¹ is lower alkyl group.

According to yet another embodiment, in the step c) compound of formula (IV) is alkoxylated using alkoxylating agent such as sodium methoxide, sodium ethoxide and so on, in presence of an alcohol such as methanol to obtain a compound of formula (Va).

According to yet another embodiment, the compound of formula (Va) obtained is converted to compound of formula (V) by treatment with an acid selected from organic or inorganic acid or mixture thereof.

The organic acid used is selected from acetic acid, formic acid, oxalic acid, and the likes. The inorganic acid used is selected from hydrochloric acid, sulfuric acid, nitric acid, phosphoric acid, boric acid, hydrofluoric acid, hydrobromic acid, perchloric acid, hydroiodic acid or Lewis acids such as boron tribromide, boron trichloride, boron trifluoride.

According to an embodiment, hydrobromic acid in acetic acid used for converting formula (Va) to compound of formula (V).

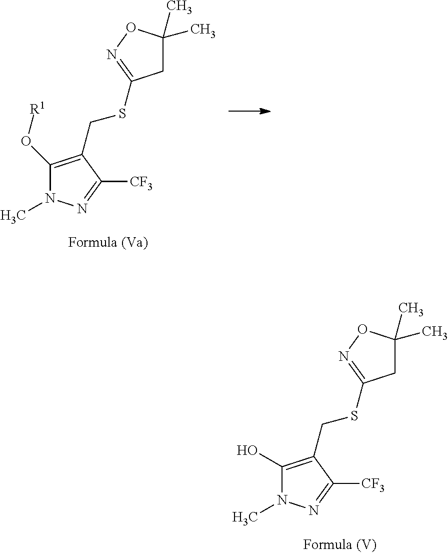

According to yet another embodiment, the compound of formula (Va) obtained is converted to compound of formula (V) by treatment with an acid in presence of a suitable solvent.

The suitable solvent used may be selected from water, an organic solvent such as chlorinated solvent like dichloromethane, dichloroethane, chloroform and the likes, ethers such as diethyl ether, tetrahydrofuran and the likes, hydrocarbons such as toluene, xylene and the like, or mixtures thereof.

According to yet another embodiment, the step c) is carried out at temperature ranging from 0° C. to 100° C.

One of the advantages of the present invention is preparation of compound of formula (V) using a simple reaction. In the process, compound of formula (IV) is alkoxylated to compound of formula (Va) which is then treated with acid to get compound of formula (V).

It was observed by the inventor of present invention that, said reaction is possible only using Formula (IV) having fluorine substitution at 5-position. Similar reaction when carried out using analogous compounds having other halogen substitution at 5-position, the reaction remains incomplete. It was observed that when said reaction was carried out using 5-chloro substituted compound i.e., compound of formula (VIII) there is no formation of compound formula (Va).

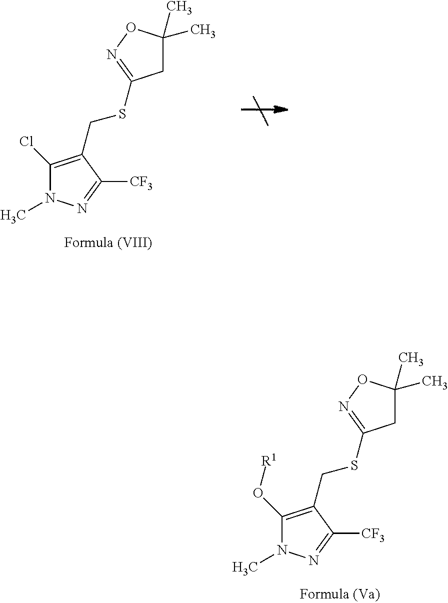

According to another embodiment, the step d) of the process comprises fluoromethylating compound of formula (V) to get compound of formula (VI), wherein step d) is carried out in presence of fluoromethylating agent such as difluorochloromethane (Freon gas) in presence of an alkaline reagent and an organic solvent.

The amount of freon gas used is in the range of 3 to 10 moles with respect to the compound of formula (V).

The alkaline reagent used can be inorganic base and/or organic base, the inorganic base is preferably one or more of potassium carbonate, sodium carbonate, potassium bicarbonate, sodium bicarbonate, potassium hydroxide and sodium hydroxide, and the organic base is preferably one or more of triethylamine, pyridine, triethylene diamine and N, N-dimethyl pyridine.

The amount of alkaline reagent used is in the range of 1.5 to 7 moles with respect to compound of formula (V).

The organic solvent used may be selected from one or more of acetonitrile, N, N-dimethylformamide, tetrahydrofuran, methanol, ethanol, isopropanol, and the like.

The amount of organic solvent used is in the range of 30 to 90 moles with respect to compound of formula (V).

According to another embodiment, the step d) is carried out at temperature ranging from 0° C. to 50° C.

According to another embodiment, the step e) of the process comprises oxidising compound of formula (VI) to get Pyroxasulfone of formula (VII) wherein oxidation is carried out in presence of an oxidizing agent.

The oxidizing agent used may be selected from organic peroxides such as m-chloroperbenzoic acid, performic acid, peracetic acid and the like; inorganic peroxides such as hydrogen peroxide, potassium permanganate, sodium periodate or oxone® (Potassium peroxymonosulfate) and the like.

The amount of oxidizing agent used is 1 to 4 moles with respect to compound of formula (VI).

According to another embodiment, the step e) oxidation is carried out in presence of an oxidizing agent in an organic solvent to obtain the Pyroxasulfone of formula (VII).

According to an embodiment, the step e) oxidation is carried out in presence of a metal catalyst.

The catalyst used can be a metal catalyst such as tungsten catalyst, molybdenum catalyst, titanium catalyst, zirconium catalyst or mixture thereof

The tungsten catalyst used may be selected from tungsten, tungstic acid, tungstic acid salt, metallic tungsten, tungsten oxide, tungsten carbide or mixtures thereof.

The tungsten catalyst such as tungsten chloride, tungsten bromide, tungsten sulfide, phospho tungstic acid or a salt thereof, tungstic acid or a salt thereof, sodium tungstate, potassium tungstate, calcium tungstate, lithium tungstate, tungsten tungstate, coordination complex of tungsten or mixture thereof, may be used.

Preferably, the tungsten catalyst used is sodium tungstate, more preferably sodium tungstate dihydrate is used.

The molybdenum catalyst used may be selected from molybdic acid, molybdates, metallic molybdenum, molybdenum carbide, molybdenum oxide, molybdenum chloride or mixtures thereof.

The molybdenum catalyst such as molybdate, sodium molybdate, potassium molybdate, ammonium molybdate, molybdate oxide (VI), molybdenum carbide, molybdenum chloride (V), molybdate sulfide (IV), phosphomolybdate, sodium phosphomolybdate, ammonium phosphomolybdate, silicate molybdate or mixtures thereof, coordination complex of molybdenum may be used.

Preferably, the molybdenum catalyst used is ammonium molybdate, more preferably ammonium molybdate tetrahydrate is used.

The titanium catalyst used may be selected from titanic acid, titanate, titanium oxide, titanium carbide, titanium chloride and mixtures thereof.

The zirconium catalyst used may be selected from zirconium oxide, zirconium carbide, zirconium chloride and mixtures thereof.

According to an embodiment, the oxidation step is carried out in presence of oxidising agent and a metal catalyst.

According to an embodiment, the oxidation step is carried out in presence of hydrogen peroxide as an oxidising agent and sodium tungstate dihydrate as a metal catalyst.

According to an embodiment, the step e) oxidation is carried out in presence of a metal catalyst and an acid.

The acid used is an inorganic acid or organic acid. The inorganic acid like sulfuric acid, hydrochloric acid, or an organic acid such as acetic acid and formic acid.

According to an embodiment, the step e) oxidation is carried out in presence of a suitable solvent selected from group comprising of halogenated hydrocarbon; ethers; amides; alcohols; ketones; nitriles; carboxylic acids; water or mixtures thereof.

The organic solvent used may be selected from halogenated hydrocarbon such as dichloromethane, chloroform, dichloroethane, carbon tetrachloride, chlorobenzene, dichlorobenzene and the like; ethers such as dioxane, tetrahydrofuran (THF), dimethoxyethane, diethyl ether and the like; amides such as N,N-dimethylacetamide, N,N-dimethylformamide, N-methyl-2-pyrrolidinone and the like; alcohols such as methanol, ethanol, propanol, isopropanol, butanol, tert-butanol and the like; ketones such as acetone, 2-butanone and the like: nitriles such as acetonitrile and the like acetic acid; water, and mixtures thereof.

According to an embodiment, the oxidation is carried out in presence of potassium peroxymonosulfate as an oxidising agent.

According to an embodiment, a process for preparation of Pyroxasulfone of formula (VII), comprising oxidising compound of formula (VI) using potassium peroxymonosulfate as oxidising agent

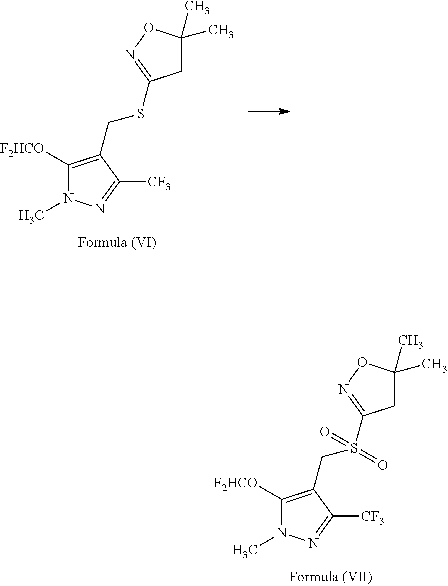 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to an embodiment, pyroxasulfone thus obtained can be further treated with aqueous base and subjected to purification by treating pyroxasulfone with alcohol or aqueous alcohol.

The base used may be selected from alkali metal hydroxides such as sodium hydroxide, potassium hydroxide and the like.

The alcohol used may be selected from methyl alcohol, ethyl alcohol, propyl alcohol, isopropyl alcohol and the like.

According to an embodiment, the purification of crude Pyroxasulfone comprises washing of the crude Pyroxasulfone with alcohol.

According to an embodiment, the purification of crude Pyroxasulfone by treatment with mixture of alcohol and water is carried out at temperature ranging from 50 to 110° C.

According to another embodiment, the step of purification of crude Pyroxasulfone further comprises cooling of reaction mixture to room temperature to isolate purified Pyroxasulfone after treatment with mixture of alcohol and water at higher temperature.

According to yet another embodiment, there is provided a process for preparation of Pyroxasulfone of formula (VII) comprising
a) condensing compound of formula (IIa)

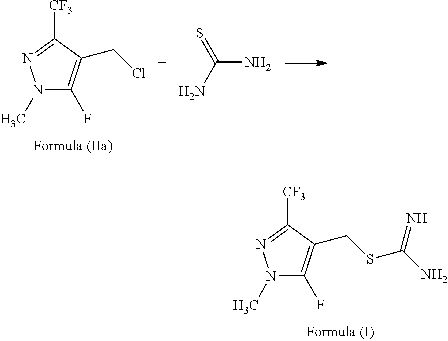

with thiourea to obtain compound of formula (I) or its salt;
b) condensing compound of formula (I) or its salt with isooxazoline compound of formula (IIIa) to get compound of formula (IV),

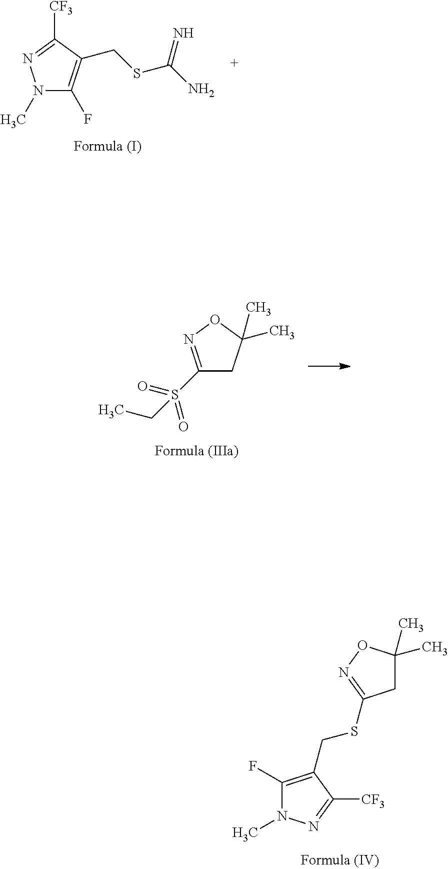 [SMILES: Cn1nc(C(F)(F)F)c(CSC(=N)N)c1F ; CCS(=O)(=O)C1=NOC(C)(C)C1 ; Cn1nc(C(F)(F)F)c(CSC2=NOC(C)(C)C2)c1F]

c) converting compound of formula (IV) to compound of formula (V)

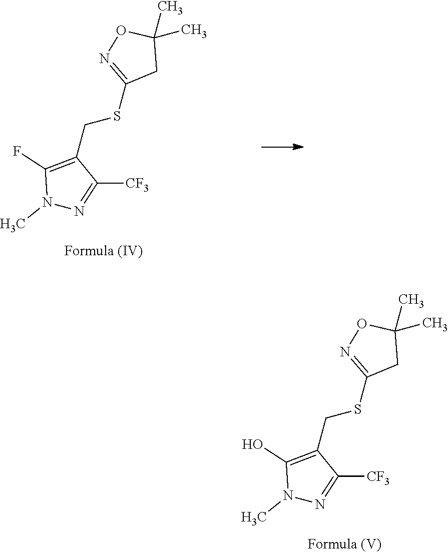 [SMILES: Cn1nc(C(F)(F)F)c(CSC2=NOC(C)(C)C2)c1F ; Cn1nc(C(F)(F)F)c(CSC2=NOC(C)(C)C2)c1O]

d) fluoromethylating compound of formula (V) to get compound of formula (VI)
   and
   Formula (V) Formula (VI)
e) oxidising compound of formula (VI) to get Pyroxasulfone of formula (VII).

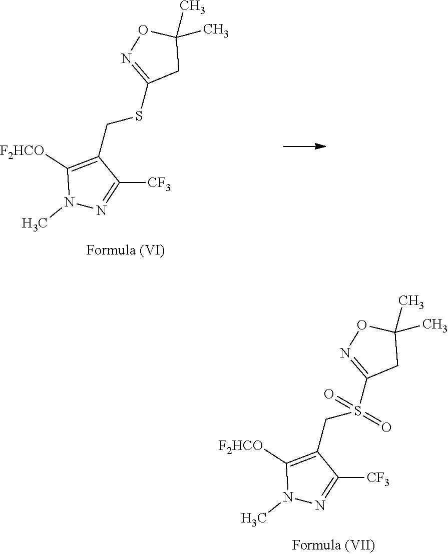 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

According to yet another embodiment, there is provided a process for preparation of Pyroxasulfone of formula (VII) comprising
a) condensing compound of formula (IIb)

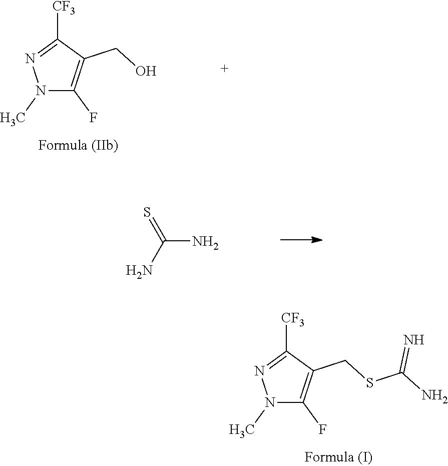

with thiourea to obtain compound of formula (I) or its salt;
b) condensing compound of formula (I) or its salt with isooxazoline compound of formula (IIIa) to get compound of formula (IV).

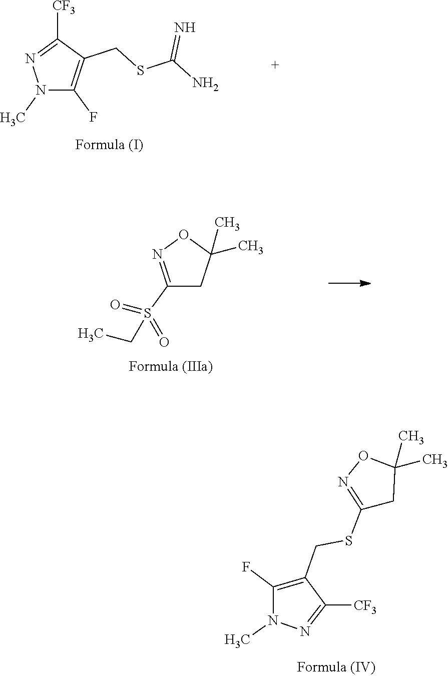

c) converting compound of formula (IV) to compound of formula (V);

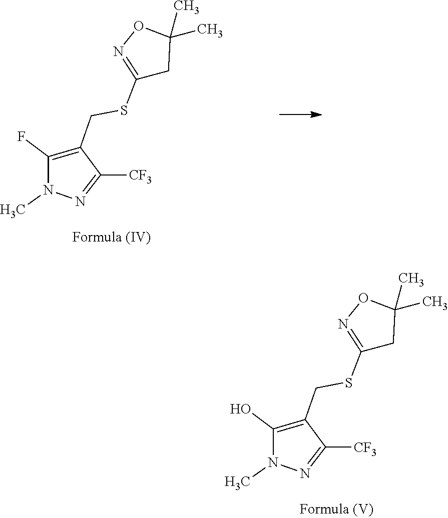

d) fluoromethylating compound of formula (V) to get compound of formula (VI) and

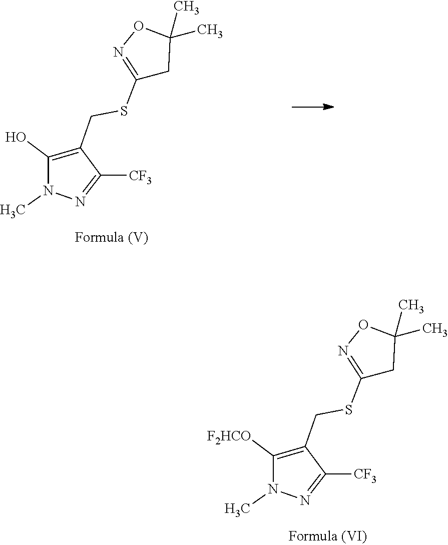

e) oxidising compound of formula (VI) to get Pyroxasulfone of formula (VII).

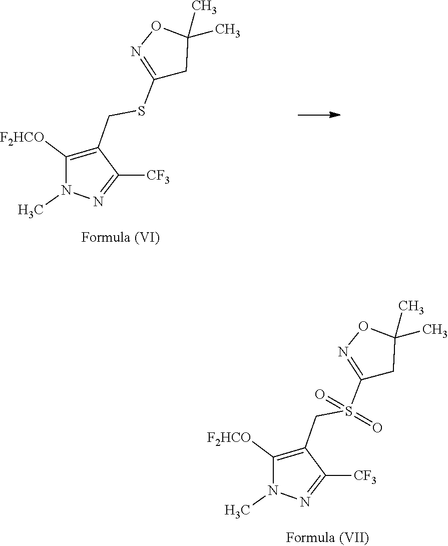

According to yet another aspect of the present invention, there is provided a process for purification of crude Pyroxasulfone comprising treating the crude Pyroxasulfone with mixture of alcohol and water at temperature ranging from 50° C. to 110° C.

According to an embodiment, there is provided a process for purification of crude Pyroxasulfone comprising treating the crude Pyroxasulfone with mixture of isopropyl alcohol and water at temperature ranging from 60° C. to 100° C.

According to yet another aspect of the present invention, there is provided a process for preparation of compound of formula (IV) comprising condensing novel compound of formula (Ia) or its salt with isooxazoline compound of formula (IIIa) to get compound of formula (IV),

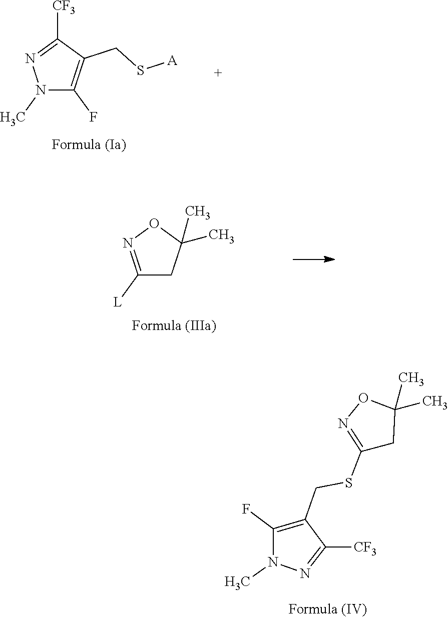

wherein A is hydrogen, —C(NH)(NH₂) or alkali metal, and L is a leaving group.

According to an embodiment, the compound of formula (Ia) wherein A is hydrogen, —C(NH)(NH₂) or alkali metal for example sodium, potassium, lithium etc, can be used.

According to an embodiment, the compound of formula (IIIa) wherein L is a leaving group such as halogen, —SO₂R¹ wherein R¹ is lower alkyl group can be used.

According to yet another aspect of the present invention, there is provided Pyroxasulfone characterized by having D₅₀ particle size value of less than about 200 μm, preferably less than about 150 μm.

According to yet another aspect of the present invention, there is provided Pyroxasulfone having bulk density of about 0.40 g/cc to 0.90 g/cc.

In another embodiment there is provided use of Pyroxasulfone in the preparation of agrochemical composition or formulation.

In another embodiment there is provided use of Pyroxasulfone prepared using the compound of formula (I) in the preparation of agrochemical composition or formulation.

In an embodiment, the agrochemical composition comprising pyroxasulfone prepared according to the present process as described herein.

According to another embodiment, the present invention provides a herbicidal composition comprising pyroxasulfone prepared according to the process as described herein and at least one agrochemically acceptable excipients.

According to another embodiment, the present herbicide composition further comprising additional herbicide. In an embodiment the additional herbicide is triazinone herbicide.

In an embodiment, the herbicidal composition comprising a combination of pyroxasulfone prepared according to the present process and a triazinone herbicide.

In an embodiment, the triazinone herbicide is selected from the group of ametridione, amibuzin, ethiozin, hexazinone, isomethiozin, metamitron, metribuzin, or trifludimoxazin. In an embodiment, the triazinone herbicide is metribuzin.

According to an embodiment, the present invention provides herbicidal composition comprising combination of pyroxasulfone prepared according to present process and metribuzin.

In an embodiment, the herbicide composition comprising pyroxasulfone prepared according to process as described herein; and at least one agriculturally acceptable excipient.

In an embodiment, agriculturally acceptable excipient/carriers can be selected from one or more diluents, emulsifiers, fillers, anti-foaming agents, thickening agents, anti-freezing agents, freezing agents, a surfactant, a preservative, a coloring agent, a pH adjusting agent, dispersing agent, wetting agent and solvent. However, it should be appreciated that any other agriculturally acceptable excipients, as known to a person skilled in the art, may be used to serve its intended purpose. In an embodiment, the agriculturally acceptable excipients are present in an amount ranging from 0.01% to 90% by weight of the total composition.

According to an embodiment, there is provided an agrochemical composition comprising Pyroxasulfone having bulk density of about 0.40 g/cc to 0.90 g/cc.

According to another embodiment, there is provided an agrochemical composition comprising Pyroxasulfone having D₅₀ particle size value of less than about 200 μm. In another embodiment, the agrochemical composition comprising Pyroxasulfone having D₅₀ particle size value of less than about 150 μm.

Pyroxasulfone prepared according to present invention can be processed into an agricultural composition of various dosage forms by conventionally known methods.

According to an embodiment of the invention, the present compositions are formulated as water dispersible granules.

Inventors of the present invention noted the ease of making compositions using Pyroxasulfone produced according to present invention.

According to an embodiment, the compositions of Pyroxasulfone obtained according to present invention are capable of dispersing quickly in water. According to an embodiment the compositions of Pyroxasulfone obtained according to present invention leads to optimum suspensibility while dispersed in water.

According to an embodiment, the composition prepared is a water dispersible granule comprising Pyroxasulfone, at least one dispersing agent and at least one wetting agent.

According to an embodiment, the dispersing agent/wetting agent used is selected from, but not limited to, group comprising of anionic, cationic or zwitterionic and/or non-ionic surface-active compounds (surfactants) or combinations thereof, preferably anionic surfactant is used.

Examples of anionic surfactants include: anionic derivatives of fatty alcohols having 10-24 carbon atoms in the form of ether carboxylates, sulfonates, sulfates, and phosphates, and their inorganic salts (e.g., alkali metal and alkaline earth metal salts) and organic salts (e.g., salts based on amine or alkanolamine); anionic derivatives of copolymers consisting of EO (ethylene oxide), PO (propylene oxide) and/or BO (butylene oxide) units, in the form of ether carboxylates, sulfonates, sulfates, and phosphates, and their inorganic salts (e.g., alkali metal and alkaline earth metal salts) and organic salts (e.g., salts based on amine or alkanolamine) or acrylic/styrene copolymers, methacrylic copolymers; linear (C₈-C₁₅) alcohol derivative and their salts; alkyl aryl sulfonates including but not limited to alkyl benzenesulfonates; alkyl naphthalene sulfonates and salts thereof and salts of ligninsulfonic acid; derivatives of alkylene oxide adducts of alcohols, in the form of ether carboxylates, sulfonates, sulfates and phosphates, and their inorganic salts (e.g., alkali metal and alkaline earth metal salts) and organic salts (e.g., salts based on amine or alkanolamine); anionic derivatives of fatty acid alkoxylates, in the form of ether carboxylates, sulfonates, sulfates and phosphates, and their inorganic salts (e.g., alkali metal and alkaline earth metal salts) and organic salts (e.g., salts based on amine or alkanolamine); alkyl ether phosphate, alkyl sulfosuccinate mono ester and diester salts.

Preferably, sulfosuccinates and their derivatives/salts; acrylic/styrene copolymers; salts of lignin sulfonic acid are used.

According to an embodiment, the composition may further comprise a defoamer. The defoamer used is selected from, but not limited to, group comprising of aqueous emulsion with polysiloxane and emulsifier, silicone oil and magnesium stearate or a suitable combination thereof.

According to an embodiment, the water dispersible granule comprising Pyroxasulfone is prepared by a process comprising:
a) mixing Pyroxasulfone with wetting agent/s and dispersing agent/s as required;
b) milling the mixture in a suitable equipment to obtain a powder having a particle size D90≤15 μm; and
c) granulating the powder by suitable means and drying the granules obtained.

According to yet another aspect of present invention, there is provided an agrochemical composition comprising Pyroxasulfone prepared according to the present process and characterized by an X-ray powder diffraction pattern exhibiting at least three peaks selected from 9.90°, 17.72°, 17.94°, 19.91°, 20.36°, 20.60°, 21.76°, 22.09°, 22.31°, 22.70°, 25.10°, 25.41°, 26.57°, 27.01°, 28.40°, and 30.18°+0.2° 2θ.

According to an embodiment, the water dispersible granule comprising Pyroxasulfone is characterized by an X-ray powder diffraction pattern exhibiting at least three peaks selected from 9.90°, 17.72°, 17.94°, 19.91°, 20.36°, 20.60°, 21.76°, 22.09°, 22.31°, 22.70°, 25.10°, 25.41°, 26.57°, 27.01°, 28.40°, 30.18°+0.2° 2θ.

### Advantages of the Present Invention

1. The present invention provides a simple, cost-effective and industrially viable alternative route to synthesis of Pyroxasulfone through preparation of intermediate compound of formula (I)
2. Said process of present invention employees use of easily available raw materials.
3. Said process of present invention proceeds through mild reaction conditions and has simpler after-treatment procedures

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### EXAMPLES

### Methods:

X-ray powder diffraction method (XPRD) pattern was carried out on
Instrument: Bruker make 2^{nd} generation D2 Phaser Powder X-Ray diffractometer;
Operated at: 30.0 kV, 10 mA;
Radiation: Cu Kα;
Mode: Reflection
Wavelength: 1.54060° A,
Scan Range: 2-40 2θ,
Step size: 0.02°

### Example 1: Preparation of [5-Fluoro-1-methyl-3-(trifluoromethyl)-1H-pyrazol-4-yl]methyl carbamimidothioate hydrochloride (1:1) Compound of Formula (I)

To 10.04 mol of ethanol was added 0.259 mol of 4-(chloromethyl)-5-fluoro-1-methyl-3-(trifluoromethyl)-1H-pyrazole (i.e., compound of formula (IIa)) and 0.316 mol of thiourea at 25-30° C. The mixture was stirred and maintained at 25-30° C. for 9 to 10 hours. After completion of reaction, the reaction mixture was concentrated under vacuum at 50-55° C. and cooled to obtain a solid mass. The solid mass thus obtained was washed with 2.32 mol of hexane and dried under vacuum to yield 89% of [5-Fluoro-1-methyl-3-(trifluoromethyl)-1H-pyrazol-4-yl]methyl carbamimidothioate hydrochloride (1:1) having HPLC purity of 77%.

### Example 2: Preparation of [5-Fluoro-1-methyl-3-(trifluoromethyl)-1H-pyrazol-4-yl]methyl carbamimidothioate hydrochloride (1:1) Compound of Formula (I)

To 8.15 mol of ethanol was added 0.210 mol of 4-(chloromethyl)-5-fluoro-1-methyl-3-(trifluoromethyl)-1H-pyrazole (i.e., compound of formula (IIa)) and 0.252 mol of thiourea at 25-30° C. The mixture was stirred and maintained at 25-30° C. for 9 to 10 hours. After completion of reaction, the reaction mixture was concentrated under vacuum at 50-55° C. and ethanol was distilled out partially. The reaction mass was then cooled to 20-25° C. to obtain solid mass. The solid mass thus obtained was filtered and washed with 1.97 mol of hexane and dried under vacuum to yield 41% of [5-Fluoro-1-methyl-3-(trifluoromethyl)-1H-pyrazol-4-yl]methyl carbamimidothioate hydrochloride (1:1) having HPLC purity of 99%

### Characterization of 5-Fluoro-1-methyl-3-(trifluoromethyl)-1H-pyrazol-4-yl]methyl carbamimidothioate hydrochloride (1:1) obtained

Colour: White
Melting point: 231.3-234.1° C.
Chloride content of compound=12.48%, which corresponds to mono HCl salt (Chloride content was calculated by HPLC)
1H-NMR value (CDOD/TMS d (ppm)): 4.41 (2H, s), 3.82 (3H, s) LC-MS (m/z): 256.2

### Example 3: Preparation of [5-Fluoro-1-methyl-3-(trifluoromethyl)-1H-pyrazol-4-yl]methyl carbamimidothioate hydrochloride (1:1) Compound of Formula (I)

To 4.66 mol of toluene was added 0.346 mol of 5-fluoro-1-methyl-3-(trifluoromethyl)-1H-pyrazol-4-yl]methanol (i.e. compound of formula (IIb)), 0.866 mol of hydrochloric acid and 0.416 mol of thiourea at 25-30° C. The reaction mixture was stirred and maintained at 90º C for 8 to 9 hours. After completion of reaction, the mixture was cooled to 15-20° C. to obtain solid mass. The solid mass was filtered and washed with 3.48 mol of hexane. The product obtained was dried under vacuum at 55° C. to yield 71% of [5-Fluoro-1-methyl-3-(trifluoromethyl)-1H-pyrazol-4-yl]methyl carbamimidothioate hydrochloride (1:1) having HPLC purity of 91% (A/A).

### Example 4: Preparation of 3-({[5-fluoro-1-methyl-3-(trifluoromethyl)-1H-pyrazol-4-yl]methyl}sulfanyl)-5,5-dimethyl-4,5-dihydro-1,2-oxazole i.e. Compound of Formula (IV)

A mixture of 0.112 mol of 3-(ethylsulfonyl)-5,5-dimethyl-4,5-dihydro-1,2-oxazole, 1.10 mol of dimethyl formamide and 0.135 mol of potassium carbonate was added to the mixture of 0.112 mol of [5-fluoro-1-methyl-3-(trifluoromethyl)-1H-pyrazol-4-yl]methylcarbamimidothioate hydrochloride, 2.91 mol of ethanol, 0.135 mol of potassium carbonate and 4.73 mol of water at 25-30° C. Then the reaction mass was heated at 50° C. for 3 hours. After completion of the reaction, ethanol was distilled out under reduced pressure followed by addition of 11.1 mol of water. The mixture was then extracted with ethyl acetate. The layers were separated, and the organic layer was distilled out to yield 72% of 3-({[5-fluoro-1-methyl-3-(trifluoromethyl)-1H-pyrazol-4-yl]methyl}sulfanyl)-5,5-dimethyl-4,5-dihydro-1,2-oxazole.

### Example 5: Preparation of 3-({[5-methoxy-1-methyl-3-(trifluoromethyl)-1H-pyrazol-4-yl]methyl}sulfanyl)-5,5-dimethyl-4,5-dihydro-1,2-oxazole i.e. Compound of Formula (Va)

0.177 mol of 30% sodium methoxide was added to the mixture of 0.071 mol of 3-({[5-fluoro-1-methyl-3-(trifluoromethyl)-1H-pyrazol-4-yl]methyl}sulfanyl)-5,5-dimethyl-4,5-dihydro-1,2-oxazole and 6.17 mol of methanol at 25-30° C. under inert atmosphere. After complete addition, the reaction mixture was heated slowly to reflux and maintained for 5-6 hours. After completion of reaction, methanol was recovered under vacuum followed by addition of 15.16 mol of water and 3.57 mol of ethyl acetate. The mixture was stirred, and layers were separated. The ethyl acetate in organic layer was distilled under vacuum to yield 88% of 3-({[5-methoxy-1-methyl-3-(trifluoromethyl)-1H-pyrazol-4-yl]methyl}sulfanyl)-5,5-dimethyl-4,5-dihydro-1,2-oxazole.

### Example 6: Preparation of 4-{[(5,5-dimethyl-4,5-dihydro-1,2-oxazol-3-yl)sulfanyl]methyl}-1-methyl-3-(trifluoromethyl)-1H-pyrazol-5-ol i.e., Compound of Formula (V)

1.278 mol of 25% HBr in acetic acid was added to 0.156 mol of 3-({[5-methoxy-1-methyl-3-(trifluoromethyl)-1H-pyrazol-4-yl]methyl}sulfanyl)-5,5-dimethyl-4,5-dihydro-1,2-oxazole, and the mixture was stirred at 25-30° C. for 4-5 hours. After completion of the reaction, acetic acid along with excess hydrogen bromide was recovered completely under vacuum and the product was precipitated by addition of 4.86 mol of water. The precipitated product was filtered, washed with 11.11 mol of water and 1.74 mol of hexane and dried to yield 72% of 4-{[(5,5-dimethyl-4,5-dihydro-1,2-oxazol-3-yl)sulfanyl]methyl}-1-methyl-3-(trifluoromethyl)-1H-pyrazol-5-ol.

### Example 7: 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole i.e., Compound of Formula (VI)

0.067 mol of sodium hydroxide was added to the mixture of 0.022 mol of 4-{[(5,5-dimethyl-4,5-dihydro-1,2-oxazol-3-yl)sulfanyl]methyl}-1-methyl-3-(trifluoromethyl)-1H-pyrazol-5-ol and 1.33 mol of acetonitrile with stirring at 22-25° C. temperature. The reaction mixture was maintained at this temperature for 1.5 hours. The mixture was then cooled to 5° C. followed by purging of 0.136 mol of freon gas at 5-15° C. within one hour. The mixture was then maintained at 22-25° C. for 3 hours. After completion of the reaction, 0.282 mol of toluene was added to the mixture followed by addition of 3.33 mol of water and 0.024 mol of 30% hydrochloric acid. The organic layer and aqueous layers were separated. The organic layer was washed with brine and the layer was distilled to yield 84% of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole

### Example 8: Preparation of Pyroxasulfone (Compound of Formula (VII))

An aqueous solution of sodium tungstate (0.0007 mol of sodium tungstate dihydrate in 0.127 mol of water) was added to the mixture of 0.021 mol of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole and 0.597 mol of acetonitrile. The mixture was slowly heated to 55° C. and then 0.058 mol of 50% hydrogen peroxide solution was added slowly by controlling temperature 75° C. within 2 hours. The mixture was maintained at this temperature for 4 hours. After completion of reaction, the reaction mass was cooled to 55-60° C. followed by addition of 0.0098 mol of 17% sodium bisulphite solution. The mixture was then stirred for 30 minutes and 0.024 mol of 48% sodium hydroxide solution was added to it. The mixture was again stirred for 5-10 minutes, and the layers were separated. The organic layer was then added to preheated 0.583 mol of water and the temperature of mixture was raised to 90-95° C. The acetonitrile in the mixture was distilled out. To the reaction mass at 80-90° C. was then added 0.221 mol of isopropyl alcohol and maintained for one hour. The mixture was then cooled 20-30° C. to precipitate product. The precipitated product was filtered, washed with mixture of isopropyl alcohol and water and dried to yield 87% of Pyroxasulfone.
Particle Size Distribution: D50=113.30 μm
Bulk density: 0.54-0.57 g/ml

### Example 9: Preparation of Pyroxasulfone (Compound of Formula (VII))

To 0.134 mol of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole was added 4.16 mol of acetic acid, 0.006 mol of sodium tungstate dihydrate and 0.42 mol of 50% hydrogen peroxide. The mixture was stirred at 25-30° C. for 8 hours. Then the temperature was increased to 50-55° C. and maintained for another 5 hours. The reaction was monitored by HPLC. After completion of reaction, the mixture was cooled to 25-28° C. and diluted with 4 mol of water. The reaction mixture was then cooled to 0° C. and maintained for 1 hour. The product was filtered out, washed with water and hexane and dried to yield 65% of Pyroxasulfone.

### Example 10: Preparation of Pyroxasulfone

To 0.080 mol of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole was added 3.42 mol of ethanol and potassium peroxymonosulfate-water mixture (0.144 mol of potassium peroxymonosulfate in 11.11 mol of water) at room temperature. The mixture was heated to 60-65° C. for 31 hours under stirring. The reaction was monitored by HPLC. After completion of reaction, the mixture was cooled to 10° C., filtered and 16 mol of water was added. The product was filtered out, washed with (1:1) mixture of water and ethanol and dried to yield 71% of Pyroxasulfone.

### Example 11: Preparation of Pyroxasulfone 85% Water Dispersible Granules

Pyroxasulfone 85% Water dispersible granules (WDG) was prepared as follows

| Sr. | | Quantity |
|---|---|---|
| No. | Composition | (% w/w) |
| 1 | Pyroxasulfone | 86.8 |
| 2 | Wetting agent | 6 |
| 3 | Defoamer | 0.2 |
| 4 | Dispersing agent | 7 |
| | Total | 100 |

Pyroxasulfone along with wetting agent/s and dispersing agent/s were taken in ribbon blender and blended for 30 minutes. After blending, the powder was milled in air jet mill to achieve milled powder having particle size D90≤15 μm. The milled powder was then post blended in ribbon blender to form homogenous mixture. This mixture and required amount of Defoamer water solution (15 to 20%) were taken in dough maker to make dough suitable for extrusion. The dough was then extruded using extruder such as basket extruder by using required aperture size of 0.5 to 0.8 mm. The extruded granules were dried in fluid bed dryer to reduce moisture content below 2% and then sieved to get final product. The final product was characterised by X-ray powder diffraction pattern.

### Example 12: Preparation of Water Dispersible Granules Comprising Pyroxasulfone+Metribuzin

Pyroxasulfone+Metribuzin Water dispersible granules (WDG) was prepared as follows:

| Sr. | | Quantity |
|---|---|---|
| No. | Composition | (% w/w) |
| 1 | Pyroxasulfone | 26.4 |
| 2 | Metribuzin | 44.0 |
| 3 | Wetting agent | 3 |
| 4 | Dispersing agent | 9 |
| 5 | Filler | q.s |
| | Total | 100 |

### Example 13: Preparation of Water Dispersible Granules Comprising Pyroxasulfone+Metribuzin

Pyroxasulfone+Metribuzin Water dispersible granules (WDG) was prepared as follows:

| Sr. | | Quantity |
|---|---|---|
| No. | Composition | (% w/w) |
| 1 | Pyroxasulfone | 13.2 |
| 2 | Metribuzin | 22.0 |
| 3 | Wetting agent | 3 |
| 4 | Dispersing agent | 9 |
| 5 | Filler | q.s |
| | Total | 100 |

1. A compound of formula (I) or its salt.

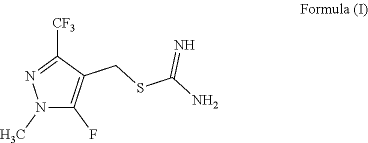 [SMILES: Cn1nc(C(F)(F)F)c(CSC(=N)N)c1F]

2. A process for the preparation of the compound of formula (I) or its salt, comprising condensing compound of formula (II) with thiourea, 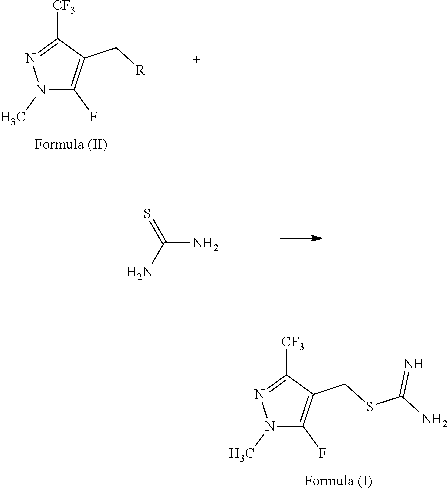
wherein R is selected from halogen or hydroxy group.

3. A process for preparation of Pyroxasulfone of formula (VII) comprising the steps of:
a) condensing compound of formula (II) with thiourea to obtain compound of formula (I) or its salt; 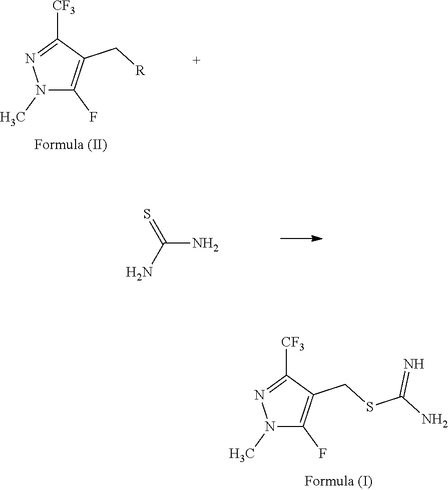
wherein R is selected from halogen or hydroxy group and
b) converting compound of formula (I) or its salt to Pyroxasulfone of formula (VII).

4. A process for preparation of Pyroxasulfone of formula (VII) comprising the steps of:
a) condensing compound of formula (II) with thiourea to obtain compound of formula (I) or its salt; 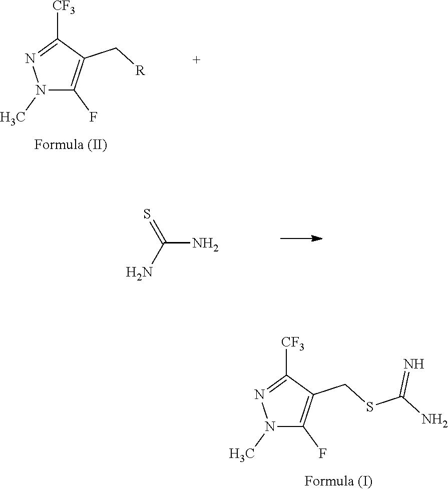
wherein R is selected from halogen or hydroxy group;
b) condensing compound of formula (I) or its salt with isooxazoline compound of formula (III) to get compound of formula (IV), 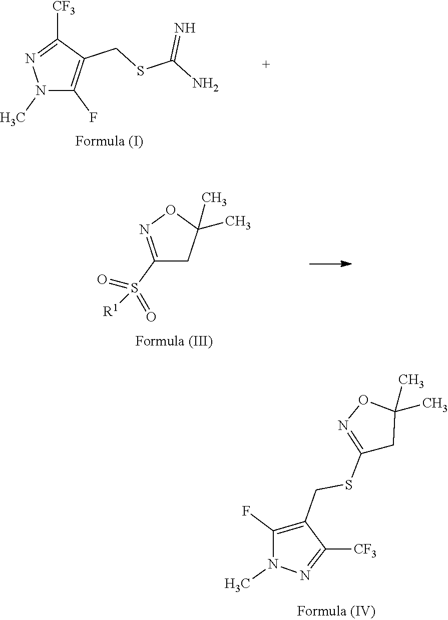
wherein R¹ is lower alkyl group;
c) converting compound of formula (IV) to compound of formula (V); 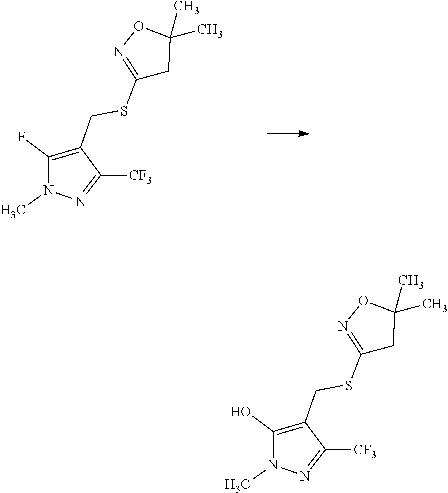
d) fluoromethylating compound of formula (V) to get compound of formula (VI); and 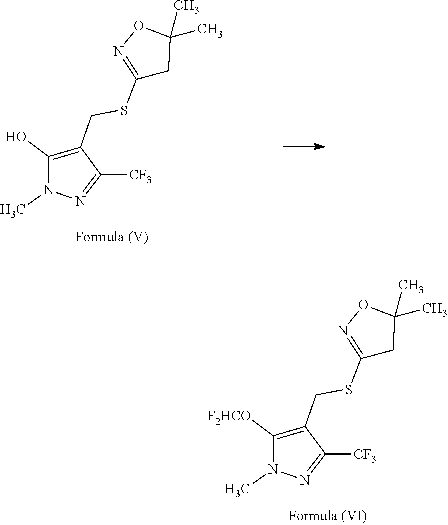
e) oxidising compound of formula (VI) to get Pyroxasulfone of formula (VII).

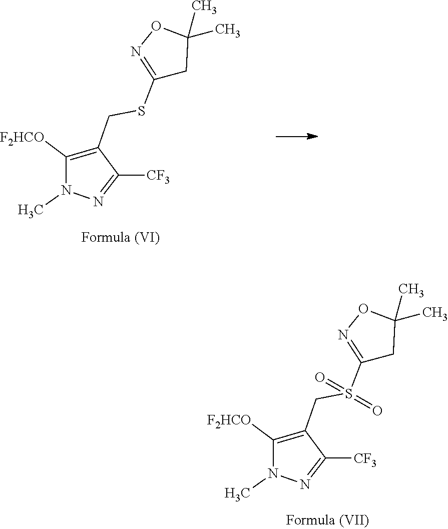

5. The process as claimed in claim 4, wherein the step a) is carried out in presence of an organic solvent.

6. The process as claimed in claim 5, wherein the organic solvent is selected from group comprising of lower alcohols, hydrocarbons, halogenated hydrocarbons, ketones and ethers.

7. The process as claimed in claim 4, wherein the step a) is carried out in presence of an organic or inorganic acid.

8. The process as claimed in claim 4, wherein the step b) is carried out in presence of a base.

9. The process as claimed in claim 4, wherein the step b) is carried out in presence of a polar solvent.

10. The process as claimed in claim 9, wherein the polar solvent is selected from group comprising of alcohols, ethers, water, dimethylformamide or mixture thereof.

11. The process as claimed in claim 4, wherein in the step c) the compound of formula (IV) is alkoxylated using an alkoxylating agent in presence of an alcohol to obtain compound of formula (Va) followed by treatment with an acid to obtain compound of formula (V) 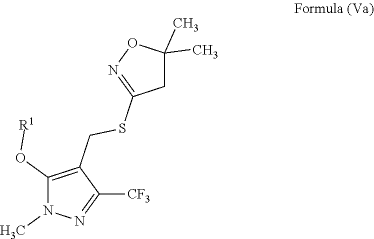
wherein R¹ is lower alkyl group.

12. The process as claimed in claim 4, wherein said oxidation is carried out in presence of an oxidizing agent.

13. The process as claimed in claim 12, wherein said oxidizing agent is an organic peroxides compound selected from m-chloroperbenzoic acid, performic acid, peracetic acid; inorganic peroxides such as hydrogen peroxide, potassium permanganate, sodium periodate or potassium peroxymonosulfate.

14. The process as claimed in claim 12, wherein said oxidizing agent is potassium peroxymonosulfate.

15. The process as claimed in claim 4, wherein said oxidation is carried out in presence of a metal catalyst.

16. The process as claimed in claim 15, wherein said metal catalyst is selected from group comprising of tungsten catalyst, molybdenum catalyst, titanium catalyst, zirconium catalyst or mixture thereof.

17. The process as claimed in claim 4, wherein said oxidation is carried out in presence of a suitable solvent selected from group comprising of halogenated hydrocarbon; ethers; amides; alcohols; ketones; nitriles; carboxylic acids; water or mixtures thereof.

18. The process as claimed in claim 4, further comprises a process for purification of Pyroxasulfone comprising treating Pyroxasulfone with mixture of alcohol and water at temperature ranging from 50° C. to 110° C.

19. Pyroxasulfone having D50 particle size value of less than about 150 μm.

20. Pyroxasulfone having bulk density of about 0.40 g/cc to 0.90 g/cc.

21. A method of using compound of formula (I) or its salt, in the process for preparation of Pyroxasulfone of formula (VII).

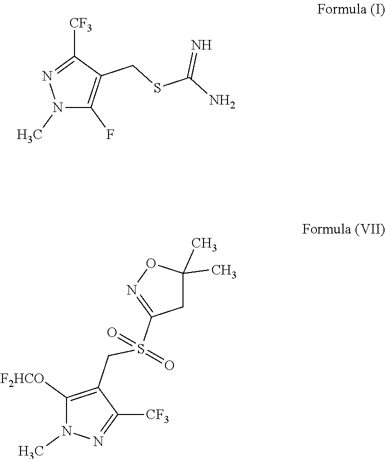 [SMILES: Cn1nc(C(F)(F)F)c(CSC(=N)N)c1F ; Cn1nc(C(F)(F)F)c(CS(=O)(=O)C2=NOC(C)(C)C2)c1OC(F)F]

22. An agrochemical composition comprising pyroxasulfone prepared by the process as claimed in claim 3.

23. The composition as claimed in claim 22, wherein said composition further comprises at least one triazinone herbicide and optionally agrochemically acceptable excipients.

24. The composition as claimed in claim 22, wherein said composition is formulated as water dispersible granules.

25. An agrochemical composition comprising pyroxasulfone prepared by the process as claimed in claim 4.
